(19) United States
(12) Patent Application Publication
(10) Pub. No.: US 2005/0137176 A1
(43) Pub. Date: Jun. 23, 2005

(54) Use of a steroidal or non-steroidal EcR receptor ligand in a cosmetic or dermatological preparation for maintaining cutaneous homeostasis

(75) Inventors: Corinne Ferraris, Paris (FR); Michelle Rathman Josserand, La Celle St Cloud (FR); Isabelle Castiel, Nice (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/018,982

(22) Filed: Dec. 21, 2004

Related U.S. Application Data
(60) Provisional application No. 60/575,440, filed on May 28, 2004.

(30) Foreign Application Priority Data
Dec. 22, 2003 (FR) 0315156

(51) Int. Cl. A61K 31/56; A61K 7/00; A61K 31/15

(52) U.S. Cl. 514/169; 514/456; 514/664; 424/401

(57) ABSTRACT

The present invention relates to the use of at least one EcR receptor ligand in a cosmetic or dermatological preparation for maintaining cutaneous homeostasis. The present invention also relates to a cosmetic treatment process for maintaining cutaneous homeostasis, which consists in applying a composition according to the invention.

### CROSS-REFERENCE TO RELATED APPLICATIONS

The present invention claims the benefit of the filing date of U.S. Provisional Application No. 60/575,440, filed May 28, 2004, the disclosure of which is hereby incorporated herein by reference.

The present invention relates to the use of at least one EcR receptor ligand in a cosmetic or dermatological preparation for maintaining cutaneous homeostasis.

The present invention also relates to a cosmetic treatment process for maintaining cutaneous homeostasis, which consists in applying a composition according to the invention.

Epidermal homeostasis is defined as being maintenance of the epidermis in a physiological state, that is to say that the renewal and differentiation of the epidermal cells must be preserved in a state of equilibrium.

Specifically, the epidermis is a keratinized multistratified epithelium that undergoes constant renewal. In order to ensure a constant thickness of the epidermis, the germinal compartment (basal layer) produces a number of cells equal to the number of cells that are removed at the surface. This fragile equilibrium is modified in the case of pathological conditions such as psoriasis. The epidermal thickness is increased and the proteins MRP8/MRP14, which are markers indicating a state of stress and/or of hyperproliferation, are then strongly expressed. The epidermis may also show signs of atrophy when the rate of cell renewal is not equal to the rate of removal of the cells at the surface.

The Applicant has found, unexpectedly, that the use of ecdysteroids makes it possible to maintain cutaneous homeostasis.

Ecdysteroids are known to play an important role not only in insects in the animal kingdom, but also in the plant kingdom. Among insects in particular, these steroid hormones play a key role in growth and reproduction.

Ecdysteroids, among which is ecdysterone or β-ecdysone ((2b, 3b, 5b,22R)-2,3,14,20,22,25-hexahydroxycholest-7-en-6-one), have thus been used for combating the signs of ageing of the skin and for improving the barrier function of the skin. Patent U.S. Pat. No. 5,198,225 specifically describes the cosmetic use of the skin structure regenerating properties of ecdysteroids for anti-ageing and in particular anti-wrinkle products. Patent FR 2 696 075 describes the use of ecdysteroids in cosmetic preparations in order to restore, preserve and/or reinforce the protective function of the epidermis. However, ecdysteroids have never been used for maintaining epidermal homeostasis.

The biological target of ecdysterone in the skin is unknown. The activity of ecdysterone on moulting and on metamorphosis in arthropods depends on the interaction of this hormone with its EcR receptor. To date, no EcR receptor has been described in mammals. To identify such a receptor, the Applicant performed immunolabelling on histological slices of reconstituted human skin with a specific EcR antibody in several insects (antibody 9B9, Developmental Biology, 1996, 180: 258-272). Analysis of the slices demonstrated labelling that was particularly present around the nuclei in the basal layer of the skin and also in the upper layers of the epidermis and in the dermis.

A first subject of the invention is thus the use, in a cosmetic or dermatological composition, of EcR receptor ligands for maintaining cutaneous homeostasis.

Another subject of the invention is a cosmetic treatment process based on this composition for maintaining cutaneous homeostasis.

Other subjects will become apparent on reading the description and the examples that follow.

The term “cutaneous homeostasis” means maintaining the physiology of the epidermis, i.e. preserving the normal rate of cell renewal and differentiation.

Cutaneous homeostasis is thus reflected by maintenance of the skin in equilibrium under physiological conditions of normality, without expression of hyperproliferation-related or stress-related markers.

In the case of atrophy of the epidermis, the cell renewal no longer compensates for the loss of cells removed at the surface. By working on a model of reconstituted skin in vitro, the Applicant has shown that the presence of an EcR receptor ligand in the culture medium results in a large increase in the thickness of the live layers of the reconstituted epidermis, without, however, reducing the horny layers, and thus prevents atrophy of the epidermis. Moreover, the increase in epidermal thickness is not associated with a hyperproliferation of cells. One of the objects of the invention is thus to maintain the skin at a thickness adapted to optimum expression of its functions, corresponding to the thickness of a healthy epidermis.

The protein markers expressed in processes of hyperproliferation and/or of stress (ultraviolet, mechanical or chemical stress) comprise the proteins MRP8 and MRP14. The detected presence of these proteins in skin cells thus represents the signature of an abnormal physiological state. Specifically, the protein MRP8 can be detected in hyperplasic epidermides and in reconstituted skin, but this protein is never found in the interfollicular epidermis of normal skin.

The addition of ecdysterone to the culture medium greatly reduces the expression of the genes coding for these proteins and also the proteins themselves, which indicates the return of these cells to a normal physiological situation.

Various EcR receptor ligands may be used. Ecdysteroids (including ecdysterone), halofenozide (RH-0345), methoxyfenoside (RH-2485), tebufenozide (RH-5992), hydrazides (Röhm & Haas Annu. Rev. Entomol. 1998, 43, 545-69), benzamide (BTBHIB from Sumitomo Chemical Co. (Agricultural Chemical Research Laboratory, Biochemical and Biophysical research Communications 1996, 227, 427-32), quercetin or coumestrol (which are both oestrogen-based flavonoids) may preferably be used.

The weight amount of the EcR receptor ligand(s) is between 0.001% and 10% and preferably from 0.01% to 5% relative to the final weight of the final composition used.

The composition used may also comprise one or more additional compounds chosen from a desquamating agent, a moisturizer and a depigmenting or propigmenting agent.

The composition according to the invention may also comprise water and/or one or more cosmetically acceptable organic solvents. These solvents may be chosen from the group consisting of hydrophilic organic solvents, lipophilic organic solvents and amphiphilic solvents, or mixtures thereof.

Among the hydrophilic organic solvents, examples that may be mentioned include linear or branched lower monoalcohols containing from 1 to 8 carbon atoms, for instance ethanol, propanol, butanol, isopropanol and isobutanol, optionally oxyethylenated polyethylene glycols, polyols such as propylene glycol, isoprene glycol, butylene glycol, glycerol, sorbitol and its derivatives, glycol ethers and propylene glycol ethers.

Amphiphilic organic solvents that may be mentioned include polyols such as propylene glycol derivatives.

Examples of lipophilic organic solvents that may be mentioned include fatty esters.

The composition according to the invention may also comprise adjuvants such as fatty substances, preserving agents, stabilizers, opacifiers, softeners, silicones, foaming agents, antioxidants, pH regulators, emulsifiers, standard hydrophilic or lipophilic gelling agents and/or thickeners, hydrophilic or lipophilic active agents, fragrances, emulsifiers, sequestering agents, polymers, acidifying or basifying agents, fillers, free-radical scavengers, ceramides, moisturizers, vitamins, surfactants, antidandruff agents, propellants, dyes or any other adjuvant usually used in cosmetics.

The amounts of these various adjuvants are those conventionally used in the field under consideration. A person skilled in the art will take care to select the optional compound(s) to be added to the composition according to the invention so as not, or not substantially, to impair the advantageous properties associated with the composition in accordance with the invention.

The compositions according to the invention are particularly suitable cosmetically and/or dermatologically and cause no irritation of the scalp, even after prolonged contact without rinsing.

The composition according to the invention comprising at least one EcR receptor ligand may be in the form of a gel, a milk, a lotion, a serum, a mask or a cream.

Another subject of the invention is a cosmetic treatment process for maintaining cutaneous homeostasis by application to an individual's skin of a cosmetically effective amount of a composition containing at least one EcR receptor ligand.

The examples that follow illustrate the invention without, however, limiting it.

### EXAMPLE 1

### Measurement of the Thickness of the Epidermis

Reconstructed epidermides were prepared according to the technique of Asselineau et al., 1985 (Asselineau D, Bernard B A, Bailly C. & Darmon M: Epidermal morphogenesis and induction of 67 kD Keratin polypeptide by culture at the air liquid interface Exp Cell Res 159: 536-539).

From the first day of exposure to the air/liquid interface, the epidermides were cultured in the presence of ecdysterone in the culture medium (1 to 1000 μg/ml). The epidermes were cultured for 7 days. The treatment was renewed every 48 hours by changing the culture medium. After treatment for 7 days, the epidermes were harvested. The histological analysis is performed after staining with HES (hematoxylin eosin saffron).

Epidermal thickness measurements were performed on histological slices. To do this, 10 measurements are taken on a field. For each sample, 5 fields are counted. These measurements are performed using a Leica Q600S image analysis system with an ×10 objective lens. The mean thickness obtained for each sample corresponds to 50 measurements taken. The set of values obtained is summarized in Table I:

TABLE I
Epidermal thickness in μm after 7 days of treatment with ecdysterone.

| Control | Ecdysterone 1000 μg/ml | Ecdysterone 10 μg/ml |
|---|---|---|
| 60.21 (9.04) | 72.05 (16) | 115 (24.07) |

The figures in parentheses represent the standard deviations.

The application of ecdysterone to the culture medium of the reconstructed epidermes results in an increase in the thickness of the epidermis at the level of the live layers. The increase in thickness of the epidermis is not accompanied by parakeratosis (nucleus in the horny layer) or by a reduction in the thickness of the horny layer. The differentiation of the epidermis is marked by the presence of numerous granular layers.

### EXAMPLE 2

### Measurements of the Cell Multiplication as a Function of the Amount of Medium

The keratinocytes are derived from a mammoplasty. These keratinocytes were isolated and then cultured in KGM medium (Clonetics) supplemented with growth factor (EGF (Epidermal Growth Factor): 10 ng/ml, 0.4 μg/ml, insulin 5 μg/ml, BPE (Bovine Pituitary Extract) 4 ml/L).

The cells are treated with ecdysterone for 8 hours to 48 hours. There is no EGF in the culture medium during the treatment with ecdysterone.

Ecdysterone was tested at the following concentrations: 1 μg/ml, 0.1 μg/ml and 0.01 μg/ml.

| Ecdysterone concentration | Treatment time | |
|---|---|---|
| in the medium (in μg/ml) | (in hours) | No of cells/flask |
| 0 | 8 | 0.8 10⁶ |
| 0.01 | 8 | problem |
| 0.1 | 8 | 0.8 × 10⁶ |
| 1 | 8 | 0.7 × 10⁶ |
| 0 | 16 | 0.8 × 10⁶ |
| 0.01 | 16 | 0.8 × 10⁶ |
| 0.1 | 16 | 1.0 × 10⁶ |
| 1 | 16 | 0.5 × 10⁶ |
| 0 | 24 | 1.1 × 10⁶ |
| 0.01 | 24 | 1.0 × 10⁶ |
| 0.1 | 24 | 1.1 × 10⁶ |
| 1 | 24 | 1.1 × 10⁶ |
| 0 | 16 | 4.4 × 10⁵ |
| 0.001 | 16 | 3.6 × 10⁵ |
| 0.01 | 16 | 2.5 × 10⁵ |
| 0.1 | 16 | 3.6 × 10⁵ |
| 0 | 24 | 5.2 × 10⁵ |
| 0.001 | 24 | 3.6 × 10⁵ |
| 0.01 | 24 | 5.7 × 10⁵ |
| 0.1 | 24 | 5.2 × 10⁵ |
| 0 | 48 | 1.9 × 10⁶ |
| 0.001 | 48 | 16 × 10⁶ |
| 0.01 | 48 | 1.6 × 10⁶ |
| 0.1 | 48 | 1.5 × 10⁶ |

After treatment for 48 hours with ecdysterone, no significant increase in the number of cells is observed, relative to the control cells.

The increase in the thickness of the live layers of the epidermis following treatment with ecdysterone is therefore not associated with hyperproliferation.

### EXAMPLE 3

### Cell Cycle Analysis

The cell cycle analysis consists in studying the DNA content of a cell population by flow cytometry. These studies were performed on normal human keratinocytes cultured as a monolayer in a defined medium (KGM-Clonetics).

The keratinocytes are derived from a mammoplasty. These keratinocytes were isolated and then cultured in KGM medium supplemented with growth factors (EGF: 10 ng/ml, 0.4 μg/ml, insulin 5 μg/ml, BPE (Bovine Pituitary Extract) 4 mL).

The cells are treated with ecdysterone for 8 hours to 48 hours. There is no EGF in the culture medium during the treatment with ecdysterone.

Ecdysterone was tested at the following concentrations: 1 μg/ml, 0.1 μg/ml and 0.01 μg/ml.

The protocol followed for the cell cycle analysis in flow cytometry is described in the following publication: Staiano-Coico L., Higgins P. J., Darzynkiewicz Z. et al; 1986 Human keratinocyte culture. Identification and stagind of epidermal cell subpopulations. J. Clin. Invest. 77, 396-404. The analysis was performed using a Facs Calibur 3C Trieur flow cytometer from the company Becton-Dickinson.

The analysis of the various phases of the cell cycle by counting in flow cytometry reveals a change in the cell population between the phases G0/G1 and G2/M as a function of the amount of ecdysterone present in the keratinocyte culture medium.

Thus, the increase in the thickness of the live layers of the epidermis following treatment with ecdysterone is the result of a change in the cell cycle.

### EXAMPLE 4

### Modulation of the Expression of Markers Involved in Hyperproliferative Phenomena: Genomic Results

Ecdysterone was applied to the medium of reconstructed epidermides under the same conditions as those of Example 1.

After treatment for 7 days, the epidermides were harvested. Analysis of the expression of messenger RNAs (mRNA) shows that the treatment with ecdysterone reduces the expression of MRP8/MRP14 mRNA by a factor of 1.7 and those of psoriasin, which is a marker associated with hyperproliferative and/or stress states, by a factor of 3.5.

Thus, ecdysterone normalizes the expression of markers associated with hyperproliferative and/or stress states of the epidermis.

### EXAMPLE 5

### Modulation of the Expression of Markers Involved in Hyperproliferation Phenomena: Protein Expression Results

Ecdysterone was added to the medium of reconstructed epidermides under the same conditions as those of Example 1.

The proteins MRP8 and MRP14 are proteins that are strongly expressed in reconstructed skin, in psoriatic skin and by keratinocytes that are hyperproliferative but that have, however, maintained the capacity to differentiate. The analysis of the protein markers MRP8 and MRP14 was performed by immunofluorescence on histological slices.

Analysis of the histological slices shows that ecdysterone restricts the expression of MRP8 and MRP14 to the upper layers of the epidermis.

### EXAMPLE 6

### Cosmetic Composition

A multiple emulsion is prepared in the following manner:

The primary emulsion is prepared by mixing the constituents of phase A at room temperature, by separately mixing the constituents of phase B at room temperature, and by slowly pouring phase B into phase A with rapid stirring. To prepare the triple emulsion, the various phases are prepared, and then phase A is slowly poured into phase B with rapid stirring. Phase C is added thereto, followed by phase D. Stirring is continued until homogenization is complete.

1. Primary emulsion:

| | |
|---|---|
| Phase A: | |
| Abil WE09 | 2.5% |
| Volatile silicone oil | 17.5% |
| Polydimethylsiloxane | 4% |
| Phase B | |
| Glycerol | 45% |
| Preserving agent | 0.8% |
| Ecdysterone | 0.2% |
| Demineralized water | qs 100% |

2. Multiple emulsion:

| | |
|---|---|
| Phase A: | |
| Primary emulsion | 20% |
| Volatile silicone oil | 10% |
| Phase B: | |
| Poly(2-acrylamido-2-methylpropanesulfonic acid) | 0.5% |
| crosslinked and neutralized with aqueous ammonia | |
| Acrylate/C₁₀-C₃₀-alkylacrylate copolymer (Pemulen | 0.3% |
| TR1) | |
| Preserving agents | 1% |
| Demineralized water | qs % |
| Phase C: | |
| Triethanolamine | 0.3% |
| Demineralized water | 2% |
| Phase D | |
| Poly(2-acrylamido-2-methylpropanesulfonic acid) | 1.5% |
| crosslinked and neutralized with aqueous ammonia | |
| Demineralized water | |

1. A method of maintaining cutaneous homeostasis in a subject in need thereof comprising applying to the epidermis a composition comprising at least one EcR receptor ligand in a cosmetically effective amount capable of maintaining said epidermis in a physiological state.

2. The method according to claim 1 wherein said EcR receptor ligand is selected from the group consisting of an ecdysteroid, halofenozide, methoxyfenoside, tebufenozide, a hydrazide, benzamide, quercetin, coumestrol and mixtures thereof.

3. The method according to claim 1 wherein said EcR receptor ligand is an ecdysteroid.

4. The method according to claim 1 wherein said EcR receptor ligand is halofenozide.

5. The method according to claim 1 wherein said EcR receptor ligand is methoxyfenoside.

6. The method according to claim 1 wherein said EcR receptor ligand is tebufenozide.

7. The method according to claim 1 wherein said EcR receptor ligand is a hydrazide.

8. The method according to claim 1 wherein said EcR receptor ligand is benzamide.

9. The method according to claim 1 wherein said EcR receptor ligand is quercetin.

10. The method according to claim 1 wherein said EcR receptor ligand is coumestrol.

11. The method according to any one of claims 1-10 inclusive, wherein said EcR receptor ligand is present in an amount of 0.001% to 10% relative to the total weight of said composition.

12. The method according to any one of claims 1-10, wherein said EcR receptor ligand is present in an amount of 0.01% to 5% relative to the total weight of said composition.

13. The method according to any one of claims 1-10, further comprising one or more additional compounds chosen from a desquamating agent, a moisturizer, a depigmenting agent and a propigmenting agent.

14. The method according to any one of claims 1-10, further comprising one or more additional compounds chosen from water, alcohol and one or more cosmetically acceptable organic solvents.

15. The method according to any one of claims 1-10 inclusive, further comprising one or more cosmetically acceptable adjuvants.

16. The method according to any one of claims 1-10 inclusive, wherein said composition is in the form of an aqueous or aqueous-alcohol solution or an aqueous or aqueous-alcoholic gel.

17. The method according to any one of claims 1-10, wherein said composition is in the form of a milk, of a gel, of a mousse, of a serum or of a lotion.

18. A method of reducing proteins in epidermis indicating hyperproliferation or stress comprising applying to the epidermis a composition comprising at least one EcR receptor ligand in a cosmetically effective amount capable of reducing expression of the genes coding such proteins or reducing the amount of such proteins to a level indicating a normal physiological state.

19. The method according to claim 18 wherein said EcR receptor ligand is selected from the group consisting of an ecdysteroid, halofenozide, methoxyfenoside, tebufenozide, a hydrazide, benzamide, quercetin, coumestrol and mixtures thereof.

20. The method according to claim 18 wherein said EcR receptor ligand is an ecdysteroid.

21. The method according to claims 18 or 19, wherein said EcR receptor ligand is present in an amount of 0.001% to 10% relative to the total weight of said composition.

22. The method according to claims 18 or 19 wherein said EcR receptor ligand is present in an amount of 0.01% to 5% relative to the total weight of said composition.

23. The method according to claims 18 or 19, further comprising one or more additional compounds chosen from a desquamating agent, a moisturizer, a depigmenting agent and a propigmenting agent.

24. The method according to claim 23, further comprising one or more additional compounds chosen from water, alcohol and one or more cosmetically acceptable organic solvents.

25. A method of increasing the thickness of the live layers of epidermis without reducing the horny layers comprising applying to the epidermis a composition comprising at least one EcR receptor ligand in a cosmetically effective amount capable of maintaining the skin at a thickness adapted to optimum expression of its functions.

26. A composition for maintaining cutaneous homeostasis in a subject in need thereof comprising: at least one EcR receptor ligand in a cosmetically effective amount capable of maintaining said epidermis in a physiological state, one or more additional compounds chosen from a desquamating agent, a moisturizer, a depigmenting agent and a propigmenting agent, and, optionally one or more additional compounds chosen from water, alcohol and one or more cosmetically acceptable organic solvents.

27. The composition according to claim 26 wherein said EcR receptor ligand is selected from the group consisting of an ecdysteroid, halofenozide, methoxyfenoside, tebufenozide, a hydrazide, benzamide, quercetin, coumestrol and mixtures thereof.

28. The composition according to claim 26 wherein said EcR receptor ligand is an ecdysteroid.

29. The composition according to claims 26 or 27, wherein said EcR receptor ligand is present in an amount of 0.001% to 10% relative to the total weight of said composition.

30. The composition according to claim 29 wherein said EcR receptor ligand is present in an amount of 0.01% to 5% relative to the total weight of said composition.
